(19) United States
(12) Patent Application Publication
(10) Pub. No.: US 2014/0171905 A1
(43) Pub. Date: Jun. 19, 2014

(54) Wearable In-Line Fluid Warmer and Battery Apparatus

(71) Applicant: James T. Lopez, Dallas, TX (US)

(72) Inventor: James T. Lopez, Dallas, TX (US)

(73) Assignee: Estill Medical Technologies, Inc., Dallas, TX (US)

(21) Appl. No.: 13/998,822

(22) Filed: Dec. 11, 2013

Related U.S. Application Data
(60) Provisional application No. 61/797,777, filed on Dec. 14, 2012.

(51) Int. Cl. A61M 5/44

(52) U.S. Cl. CPC A61M 5/445
USPC 604/507; 604/114; 29/825

(57) ABSTRACT

An inline fluid warming apparatus that has a fluid warmer portion and a battery portion for powering the fluid warmer portion. The fluid warmer portion has a housing that connects directly to a housing of the battery portion by an attachment means. The attachment can be a rail system or a snap attachment or any other secure means for attaching the two housings together. The fluid warmer portion has power contacts that engage with power contacts on the battery when the two portions are engaged. When the power contacts are engaged, the battery portion provides power to the fluid warmer portion to warm fluid traveling through the fluid warmer. The apparatus is placed directly onto an arm or leg of a patient. A band or adhesive may be used to hold the apparatus in place against the skin of the patient.

This is a non-provisional application claiming priority under 35 USC 119(e) of provisional application Ser. No. 61/797,777, filed Dec. 14, 2012.

### BACKGROUND OF THE INVENTION

1. Field of the Invention

The present system relates to an apparatus that is intended to warm fluids to body temperature for introduction to a patient. The apparatus includes a fluid warmer portion and a battery portion that connect together through a sliding rail system. The apparatus is wearable on the arm or leg of the patient and may also be attached to a patient by use of an adhesive.

2. Brief Description of Related Art

Fluid introduced intravenously should be warmed to a temperature approximating body temperature to prevent the lowering of core body temperatures. Some intravenous fluids are shelf stable and are stored at ambient temperatures which are very often 75 degree F. or below (more than 20 degree F. below normal body temperature). Other intravenous fluids must be stored at refrigerated temperatures in the 32 degree F. to 40 degree F. range (more than 50 degree F. below core temperature). In emergency situations, such intravenous fluids may necessarily be introduced at refrigerated temperatures directly into the body through I.V. tubes. Introduction of such liquids at these refrigerated temperatures, however, presents a substantial risk for injurious chill or shock to the body.

A variety of devices have been developed to address the issue of the warming of intravenous fluids. Current systems generally deliver fluid to the body through an elongated I.V. tube that connects the fluid warmer to the patient. These devices are either hung from an apparatus or are placed on a surface near the patient.

### SUMMARY OF THE INVENTION

The invention is directed to an inline fluid warming apparatus that has a fluid warmer portion and a battery portion for powering the fluid warmer portion. The fluid warmer portion has a housing that connects directly to a housing of the battery portion by an attachment means. The attachment can be a rail system or a snap attachment or any other secure means for attaching the two housings together. The fluid warmer portion has power contacts that engage with power contacts on the battery when the two portions are engaged. When the power contacts are engaged, the battery portion provides power to the fluid warmer portion to warm fluid traveling through the fluid warmer. The fluid to apparatus is intended to be placed directly onto an arm or leg of a patient. A band may be used to hold the apparatus in place against the skin of the patient. However, in cases where a patient's injuries are so severe that the fluid warmer cannot be attached to an arm or leg, the fluid warmer may be attached to the body using an adhesive.

### BRIEF DESCRIPTION OF THE FIGURES

FIG. 1 is a perspective view of the apparatus of the invention showing a first end;

FIG. 2 is a perspective view of the apparatus of the invention showing a second end;

FIG. 3 is a side overhead view of the apparatus of the invention showing the battery portion and the fluid warmer portion partially engaged;

FIG. 4 is a topside view of the fluid warmer portion of the apparatus;

FIG. 5 is a topside view of the battery portion of the apparatus;

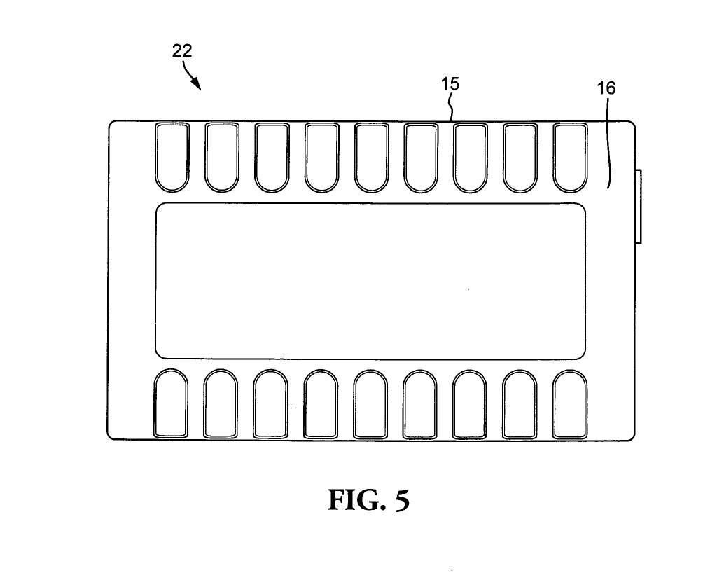

FIG. 6 is a side view of the apparatus of the invention showing the battery portion and the fluid warmer portion fully engaged;

FIG. 7 is an end view of the apparatus of the invention showing the battery portion and the fluid warmer portion fully engaged;

FIG. 8 is a perspective view of the apparatus with the battery portion and fluid warmer portion partially engaged and with an armband; and

FIG. 9, is an end view of the apparatus of the invention having an accessory mounted between the fluid warmer and the battery portion.

### DETAILED DESCRIPTION

Definitions:

The term fluid warmer means a cartridge having a housing containing a heater. The cartridge may have an internal fluid flow path that is either permanent or removable. The heater warms the flow path, in turn, warming fluid as it flows in and through the fluid warmer and out to a patient. Generally the flow path is connected at one end thereof to an IV tube that is connected to a patient and the flow path is connected at a second end thereof to a fluid source.

The term battery means any type of suitable battery that is capable of powering a fluid warmer. Examples of such batteries include but are not limited to a lithium ion or lithium polymer batteries.

The term IV tube means in intravenous tube.

The terms power connector means any type of power receiving opening that receives a power cable to power the respective device, i.e. the fluid warmer.

The term ledge means an elongated bar that supports a strap or band.

The term adhesive means an adhesive pad or other adhesive that connects the fluid warmer to the skin of a patient to hold it securely in place and also has the property of being medically compatible with skin and medical applications.

The term housing means any sturdy protective housing that safely protects the contents within. The housing material may be plastic, metal, or other sturdy material.

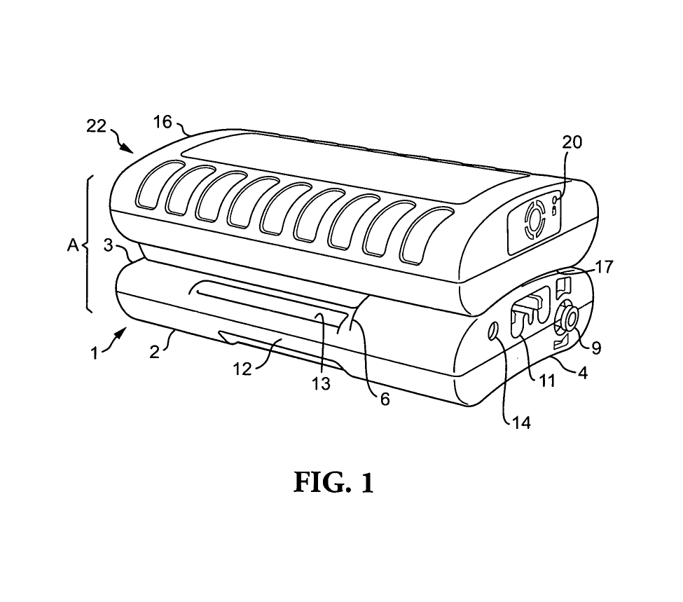

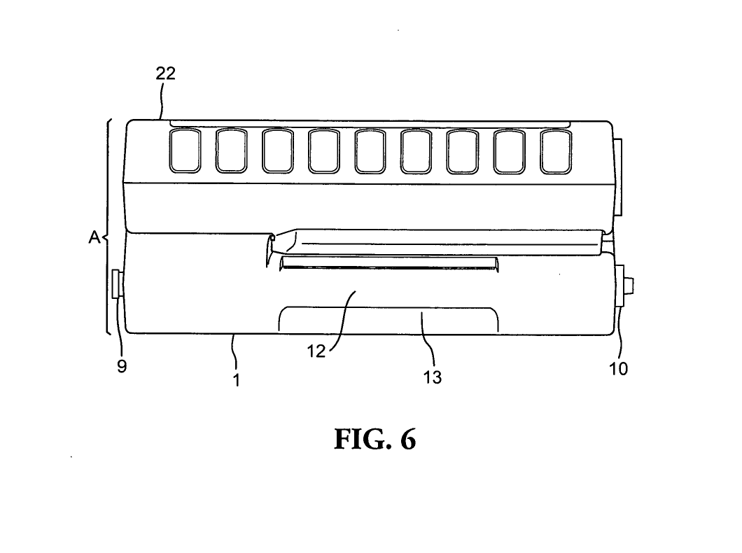

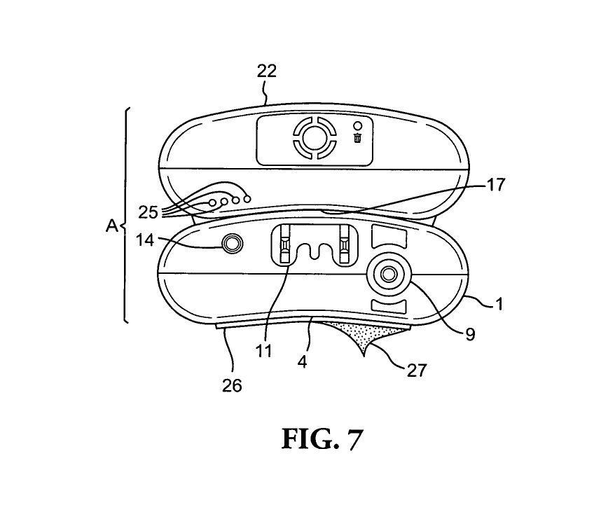

As shown in FIG. 1, the apparatus of the invention is directed to a combination fluid warmer portion 1 and a battery portion 22 that fit together to form the apparatus A. The apparatus A is also shown in FIGS. 1-3 and 6-8. The fluid warmer has a substantially rigid housing 2 having an upper surface 3 and a concave bottom surface 4. It is preferable that the upper surface 3 is convex so that a cross section of the fluid warmer is substantially kidney shaped (See FIG. 7). However, any shape is envisioned with the proviso that it can be attached to the arm or leg of a patient. The concave bottom surface 4 is preferable because it conforms to the shape of the arm or leg of a patient, however, the bottom surface could be planar. In the case where adhesive is used as the attachment means, the bottom surface could be substantially flat.

The rigid housing 2 encloses a heater and fluid flow path (not seen). The housing further has an IV inlet 9 and an IV outlet 10.

The preferred shape of the bottom surface 4 of the fluid warmer housing 2 is designed to comfortably fit onto and conform to the generally curved shape of a user's arm. The placement of the fluid warmer portion and/or full apparatus on a user's arm using an arm band attachment is advantageous because a much shorter IV tube is needed to connect the fluid warmer to the user. A shorter IV tube is preferable to a long IV tube because it has less heat loss from the warmed fluid exiting the fluid warmer. It is also cheaper to use shorter fluid paths, as less IV tubing is needed.

The fluid warmer portion 1 has an alternate or secondary female power connector 11 for receiving a power cable as a secondary means for running the fluid warmer portion. This could be used in the scenario when the battery portion 22 is fully discharged and there are no replacement battery portions or means for charging the battery portion available. In that scenario, an alternate power connector enables the user to use alternative batteries or power sources to power the fluid warmer portion.

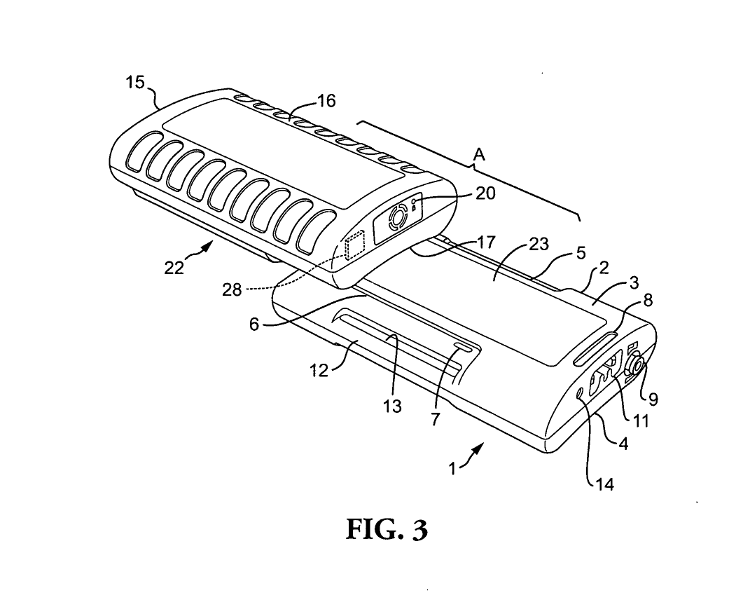

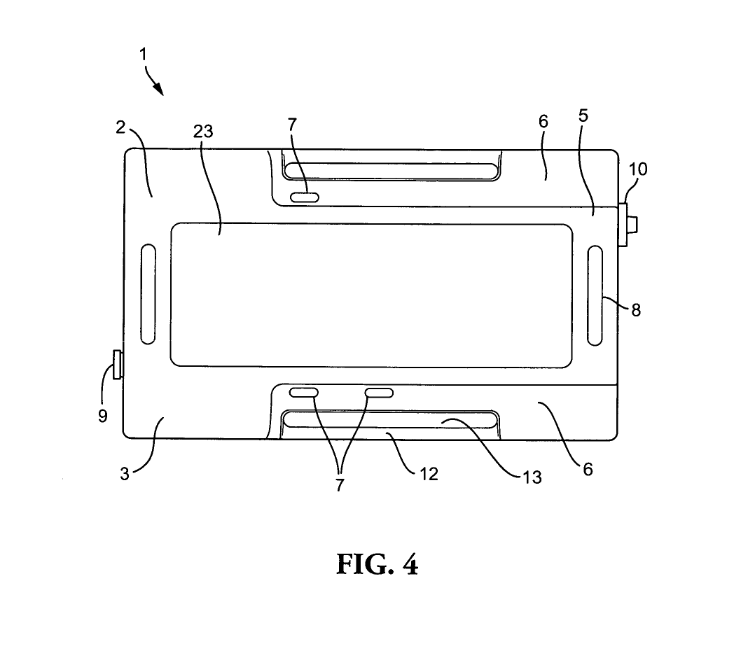

The fluid warmer potion 1 has a protruding elongated rail 5 on an upper surface 3 thereof. The rail 5 is formed by parallel-elongated recesses 6 on the upper surface 3 of the fluid warmer portion 1. Located in the elongated recesses 6 are power contacts 7 (FIGS. 3-4). Preferably two power contacts 7 (FIG. 4) are positioned in one of the parallel elongated recess and a third power contact 7 is located in the other of the elongated recesses 6 (FIG. 3). However, there can be other power contact configurations or numbers for electrically connecting the fluid warmer portion to the battery portion, with three being the preferred number.

The rail 5 is for communication and engagement with the battery portion 22 of the apparatus. The three power contacts 7 are for contacting complimentary power contacts (not shown) in the battery portion 22 of the apparatus. The third power contact is a safety contact. When the power contacts 7 of the fluid warmer portion engage the power contacts of the battery portion (not shown), the fluid warmer portion receives current to heat the heater inside of the fluid warmer.

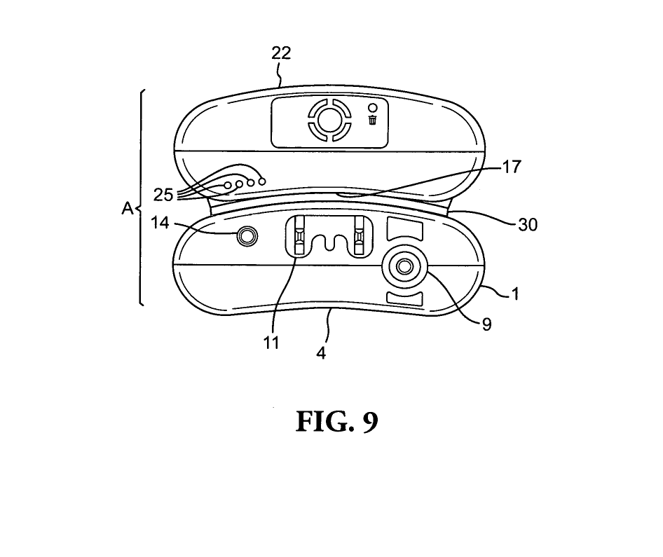

The rail system is available as a conduit for other accessories 30, as shown in FIG. 9, that may fit between and attach to the battery and the fluid warmer. The accessory(s) would have their own power contacts for engaging with the power contacts on the battery portion and the fluid warmer portion. Non-limiting examples of accessories include communication devices, GPS, and patient monitoring devices.

The rail system as described above is most preferred and believed to be most efficient. However, in an alternative embodiment, a snap on system can be employed wherein the fluid warmer portion has a means for snapping onto or with the battery portion. When the two portions are snapped together in full engagement, power and communication contacts are engaged between the battery portion and the fluid warmer portion.

The battery portion 22 has a housing 15. The housing 15 has an upper surface 16 and a lower surface 17. The upper surface 16 is preferably convex and the lower surface 17 is preferably concave so that a cross section of the battery is generally kidney shaped as shown in the figures. However, the shape of the lower surface of the battery only needs to be complimentary for engaging with and to the upper surface of the fluid warmer. Thus, the lower surface of the battery can be planar, for example, if the upper surface of the fluid warmer is planar. The shape of the battery must promote proper fit and engagement with the rails 5 and contacts 7 of the fluid warmer. The lower surface of the battery portion has power contacts (not shown) for engaging with the power contacts 7 of the fluid warmer portion.

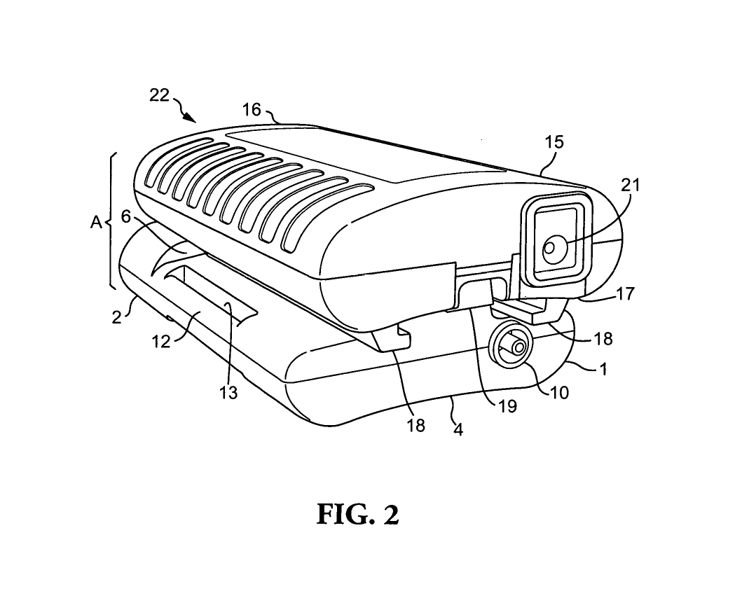

As shown in FIG. 3, the battery portion 22 slides onto one end of the fluid warmer portion by engaging the rail 5 of the fluid warmer portion within the parallel tracks 18 of the battery portion. A spring latch 19 (FIG. 2) on the battery portion engages with a groove 8 in the fluid warmer portion to hold the battery portion and the fluid warmer portion together. The spring latch 19 can be released when desired to remove the battery from the fluid warmer portion. A new fluid warmer portion can then be engaged with the same battery portion. Alternatively, the spring latch may be on the fluid warmer portion and the groove may be on the battery portion without taking away from the spirit of the invention.

The fluid warmer portion has an optional power indicator light 14 to communicate when the fluid is warmed to the manufacturer's specification. The battery portion has a “throw away” indicator light 20 to communicate when the battery is no longer viable. The battery may also have a series of indicator lights 25 that communicate the condition of the battery or other information such as, for example, a one illuminated light for “low battery” charge, two illuminated lights for “50% battery” charge, three illuminated lights for “75% battery” charge and a four illuminated lights for “fully charged.”

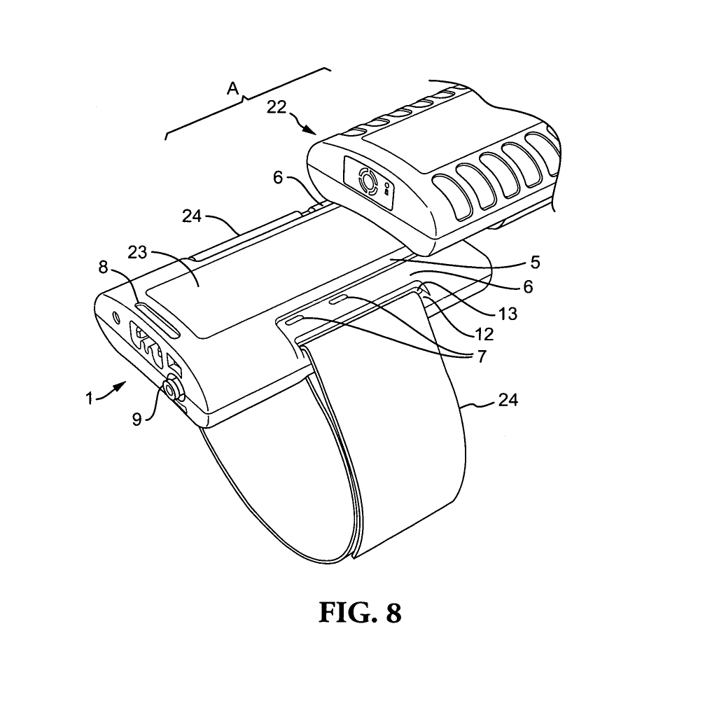

In one embodiment, the fluid warmer portion has one or more openings 13 for receiving an armband 24 (FIG. 8). The housing 2 defines the openings and has a ledge 12 for attachment of the armband 24. It is preferred that the housing has two ledges on opposite sides of the housing for receiving opposing ends of an armband. The armband 24 should be of sufficient length to wrap around a patient's arm. It is also envisioned to have an armband large enough to wrap around a leg or torso of a patient. The armband can be adjustable and can be made of cloth, elastic, rubber or any material that can conform and enwrap an arm of a patient. Hook and loop (Velcro®) attachment means may be provided.

Alternatively, the fluid warmer housing 2 may have an adhesive pad 26 attached to the lower surface of the fluid warmer as shown in FIG. 7 for affixing the fluid warmer to a patient. A peel-away strip of paper 27 can be attached to the adhesive pad to permit a care-giver or patient to easily access the adhesive and apply the fluid warmer to the body.

The battery is preferably reusable and can be recharged. The battery can be charged by multiple power sources via the power jack 21. An elongated cable (not shown) is used to connect the power jack 21 to an external power source for charging. A controller 28 inside of the battery determines what power source(s) is attached and correctly charges the battery portion based on the power source or preferred power source.

The battery may also be disposable and non-rechargeable. As such a power jack is not mandatory for the battery.

The fluid warmer portion or the internal contents thereof, i.e. fluid path, is disposable. The advantage of a rechargeable battery and a disposable fluid warmer is that one battery portion can be used to power multiple fluid warmer portions.

The fluid warmer portion 1 may also have a label 23 with indicia symbols for guiding a user on how to connect the battery portion to the fluid warmer portion.

The attachment of the battery portion to the inline fluid warmer portion via the rail system with power contacts eliminates the need for a power cable attachment between the two portions. This reduces the weight of the apparatus as a unit. It further provides an efficient and compact device.

In another embodiment of the invention, the battery portion and fluid warmer portion can be a single unit enclosed by a single housing. The single housing shape would be of a size and shape to attach and fit comfortably to the arm or leg of a user. The single housing would have a means for attaching to the arm or leg such as a strap or armband.

In still another embodiment, the apparatus is not necessarily body wearable but has the battery portion and fluid warmer portion that are not necessarily shaped to be attached to a body limb, i.e. flat, circular, etc.

### EXAMPLES

### Example 1

### Embodiment Having Two Portion Apparatus, Attached to Arm or Leg

In use a user or health care giver attaches the fluid warmer portion to the battery portion by sliding the parallel tracks of the battery portion onto the rail of the fluid warmer portion so that the power contacts of the battery portion engage with the power contacts of the fluid warmer portion. The full engagement includes the step of engaging the spring latch of the battery portion into the groove of the fluid warmer portion.

The battery portion provides the current to power the internal circuitry of the fluid warmer and the fluid warmer provides heat to fluid entering the fluid warmer from the inlet. The user connects the fluid warmer portion to a fluid source bag via the inlet. The full apparatus including battery portion and fluid warmer portion are attached to an arm or leg of a patient via the armband or adhesive. This is accomplished by placing the concave lower surface of the fluid warmer against the arm or leg for a snug fit. The user then connects the outlet of the fluid warmer to an IV tube that is connected to the patient. The fluid warmer then provides warmed fluid to the patient from the external fluid source. The fluid is blood, serum, saline or any type of fluid desired to be provided by the health care provider.

When the fluid administration to the patient is complete, the apparatus is removed from attachment to the patient. The fluid warmer portion is then discarded. The battery portion is now ready for recharging if needed and attachment to another fluid warmer portion.

### Example 2

### Embodiment Attaching Male Power Connector of Fluid Warmer to Alternative Power Source

In use the user or health care giver attaches the female power connector of the fluid warmer to a secondary source of power via a cable. The secondary source of power can be any type of battery or suitable power source. The user does not have to use the battery portion 22 but only the fluid warmer portion 1. Alternatively, the user may leave the battery attached to the fluid warmer and the battery will be charged or recharged by using the male power connector attached to the fluid warmer female power connector. The user then connects the fluid warmer portion to a fluid source bag via the inlet. The apparatus is attached to an arm or leg of a patient by placing the concave lower surface of the fluid warmer against the arm or leg for a snug fit. The armband is wrapped around the patient's appendage to hold the lower concave surface of the fluid warmer against the appendage. The user then connects the outlet of the fluid warmer to an IV tube that is connected to the patient. The fluid warmer provides warmed fluid to the patient from the external fluid source.

### Example 3

### Embodiment Attaching a Single Housing Apparatus to a Patient

In the embodiment where the apparatus has a single housing that houses the battery and fluid warmer, the user or health care giver attaches the apparatus to the patient using a band. The battery provides the current to power the internal circuitry of the fluid warmer, thereby heating fluid entering the fluid warmer from the inlet. The user connects the fluid source bag via the inlet.

### Example 4

### Embodiment wherein the Apparatus is not Attached to the Patient Directly

In use a user or health care giver attaches the fluid warmer portion to the battery portion by sliding the parallel tracks of the battery portion onto the rail of the fluid warmer portion so that the power contacts of the battery portion engage with the power contacts of the fluid warmer portion. The full engagement includes the step of engaging the spring latch of the battery portion into the groove of the fluid warmer portion. The apparatus is attached nearby the patient's body rather than being attached directly to the body. This may be desirable in a situation such as a burn patient.

What is claimed is:

1. An inline fluid warming apparatus comprising:
a. a fluid warmer portion having a housing with an attachment means and
b. a battery portion having a housing with attachment means complimentary to said fluid warmer attachment means for electrically engaging said fluid warmer portion to said battery portion, wherein said fluid warmer housing connects directly to the battery warmer housing.

2. The apparatus of claim 1, wherein said fluid warmer portion has a rail system with power contacts and said battery portion has a rail system with power contacts for sliding engagement with said fluid warmer portion to electrically connect said two portions.

3. The apparatus of claim 1, wherein said apparatus further comprises an attachment means for connecting the apparatus to an arm, leg or other part of a patient.

4. The apparatus of claim 3, wherein said attachment means comprises a band or adhesive pad.

5. The apparatus of claim 4, wherein said adhesive pad has a peel away strip for exposing said adhesive.

6. The fluid warmer of claim 1, wherein said fluid warmer portion has an IV inlet and an IV outlet for permitting attachment to IV tubing.

7. The fluid warmer of claim 1, wherein said fluid warmer has a bottom surface that is substantially concave for comfortably fitting onto an arm or leg of a patient.

8. The apparatus of claim 1, further comprising a power jack in said battery portion for receiving an elongated cable to charge said battery portion.

9. The apparatus of claim 1, wherein said fluid warmer has a snap-on engagement means for snapping onto a complimentary snap-on engagement means on said battery portion.

10. The apparatus of claim 1, wherein said battery portion has a spring latch for engaging said fluid warmer portion securely to said battery portion or said fluid warmer portion has a spring latch for engaging said battery portion securely to said fluid warmer portion.

11. The apparatus of claim 1, wherein said fluid warmer portion has a female power connector for receiving a power cable from an external power source to charge the fluid warmer portion and/or the battery portion.

12. The apparatus of claim 1, wherein said fluid warmer portion housing defines one or more openings for receiving an armband, whereby said armband is capable of attaching said fluid warmer portion directly to an appendage of a patient.

13. The apparatus of claim 1, wherein said fluid warmer portion has a power indicator light for indicating electrical attachment to said battery portion or to an external power source.

14. The apparatus of claim 1, wherein said battery portion has a controller that determines what power source and/or preferred power source that is attached and correctly charges the battery portion.

15. The apparatus of claim 1, wherein said battery portion has one or more indicator lights for indicating the condition or charge level of the battery portion.

16. The apparatus of claim 1, wherein said fluid warmer portion, internal contents of the fluid warmer, and/or battery are disposable.

17. The apparatus of claim 1, further comprising an accessory device mountable between the battery and the fluid warmer.

18. A fluid warming apparatus comprising a housing, said housing enclosing a battery, a heater and a fluid warmer flow path, wherein said housing has a lower surface with a concave shape for fitting comfortably onto an appendage.

19. A method of attaching a battery to a fluid warmer comprising:
sliding the battery portion of claim 2 onto a fluid warmer portion of claim 2 by engaging said rail system until said power contacts on said fluid warmer engage with said power contacts on said battery.

20. A method of warming fluid to be delivered to a patient intravenously comprising:
providing a fluid warmer of claim 1;
providing a fluid from a fluid source;
transferring the fluid from the fluid source to the fluid warmer;
warming the fluid with the fluid warmer to provide warm fluid;
connecting the fluid warmer via an i.v. tube to a patient's vein; and
transferring the warm fluid to the patient.
